(19) United States
(12) Patent Application Publication
(10) Pub. No.: US 2009/0048665 A1
(43) Pub. Date: Feb. 19, 2009

(54) Fastening Device

(75) Inventors: Yehuda Miron, Kfar-Haim (IL); Yuri Sudin, Modiin (IL); Ron Karmeli, Haifa (IL)

Correspondence Address: THE NATH LAW GROUP, 112 South West Street, Alexandria, VA, 22314, US

(73) Assignee: VAYRO LTD., Kfar-Haim (IL)

(21) Appl. No.: 12/224,601

(22) PCT Filed: Mar. 5, 2007
(86) PCT No.: PCT/IB2007/000511

§ 371 Date: Sep. 2, 2008

Related U.S. Application Data
(60) Provisional application No. 60/778,367, filed on Mar. 3, 2006.
(60) Provisional application No. 60/858,359, filed on Nov. 13, 2006.

(51) Int. Cl. A61F 2/44; A61B 17/08

(52) U.S. Cl. 623/1.36; 606/151

(57) ABSTRACT

A fastening device is provided to be associated with a first object, for the attachment of the object to a second object. The device comprises at least one fastening element having a base portion and at least one securing portion. The fastening element is originally produced with its securing portion having a first orientation relative to the base portion and with a possibility to bring the securing portion into a second orientation relative to the base portion, to allow the at least one securing portion to penetrate the second object being exposed thereto while staying in the second orientation, and being adapted to change the second orientation into the first orientation to provide the attachment.

### FIELD OF THE INVENTION

This present invention relates to fastening devices and methods for the attachments of objects to each other, in particular to medical fastening devices and methods, especially those used for anastomosis.

### BACKGROUND OF THE INVENTION

Fastening devices of the kind to which the present invention refers generally comprise elements which are most often adapted to penetrate object or objects to be attached by them and their orientation may be changed during the penetration. Examples of different connectors of this kind are presented below.

U.S. Pat. No. 5,597,378 discloses medical devices incorporating SIM alloy elements. Medical devices which are currently proposed to use elements made form shape memory alloys may be improved by the use of stress-induced martensite alloy elements instead. The use of stress-induced martensite decreases the temperature sensitivity of the devices, thereby making them easier to install and/or remove.

U.S. Pat. No. 5,207,695 discloses an aortic graft, implantation device, and method for repairing aortic aneurysm. An aortic graft and system and method for implanting the aortic graft are provided. The aortic graft comprises a substantially cylindrical graft material with attachment means which comprise a plurality of post and hook assemblies which are implanted with a double catheter system to penetrate the aorta wall above and below the aneurysm to provide firm attachment of the aortic graft and, thereby, exclude the aneurysm from the circulatory system.

U.S. Pat. No. 7,081,132 discloses a flexible barb for anchoring a prosthesis. A barbed medical prosthesis is disclosed in which the barb includes a basal portion comprising a point of union with the substrate of origin (such as a strut), an anchoring portion adapted to embed into tissue, and a stress-dispersing portion located between the basal and anchoring portions. In one embodiment the stress-dispersing portion comprises a helical coil having a free winding that is unattached to the strut from which the barb extends. In another embodiment, the stress-dispersing portion comprises a series of bends or curves formed in the barb proximate to the point of union with the strut. The barb can be mechanically attached and/or soldered to the prosthesis, or integrally formed therefrom. A second barb portion and stress-dispersing portion, may also extend from the basal portion.

US 20050256531 discloses an apparatus and methods implant a fastener in a targeted body region, e.g., a hollow body cavity or an intraluminal space. The apparatus and methods deploy in the targeted body region a fastener attachment assembly that carries an actuated member. The actuated member is selectively operable to generate an implantation force to implant a fastener into tissue within the targeted body region. The fastener can be implanted, e.g., to secure a prosthesis, e.g., an endovascular graft. The systems and apparatus apply a resolution force at or near the actuated member, thereby making possible a stable and dependable catheter-based fastening platform.

US 20060030921 discloses a method and apparatus for a securement device useful for the treatment of aneurysms includes a hub and, in one aspect, a plurality of arms or spikes in a star pattern extendable therefrom and into engagement with a blood vessel wall. The securement device may be deployed to anchor a secondary device, such as an exclusion device for example a stent graft, in position in a flow lumen and thereby prevent the migration of the exclusion device in the flow lumen. The arms may be positioned to penetrate through the exclusion device and thence into the flow lumen wall to provide such securement.

U.S. Pat. No. 6,358,258 discloses a device and method for performing end-to-side anastomosis. Devices, methods, and kits are provided for suturing an end of a first body duct to a hole in the side of a second body duct. The present devices and methods are used to simplify the suturing procedure and thus reduce operating time. In one embodiment, the present device includes a structure for holding the end of the first body duct and positioning the end adjacent to the hole in the side of the second body duct. The structure of the device is typically a shaft having a surface adapted to receive the first body duct. A plurality of needles are arranged on the structure to be advanced along a plurality of paths. Each needle path first passes radially into and forwardly out of the end of the first body duct and into the hole of the second body duct. The path then everts so that the needles and associated sutures will pass outwardly through tissue peripheral to the hole when the end of the first body duct is on the structure adjacent to the hole in the second body duct. The needles preferably travel along such paths when they are advanced forward. In one embodiment, the device uses a J-shaped tube for guiding one of the needles along the desired path. In another embodiment, shape-memory needles having an arcuate profile are used to create the desired path.

U.S. Pat. No. 7,048,747 discloses a device and method for performing end-to-side anastomosis. Devices, methods, and kits are provided for suturing an end of a first body duct to a hole in the side of a second body duct. The present devices and methods are used to simplify the suturing procedure and thus reduce operating time. In one embodiment, the present device includes a structure for holding the end of the first body duct and positioning the end adjacent to the hole in the side of the second body duct. The structure of the device is typically a shaft having a surface adapted to receive the first body duct. A plurality of needles are arranged on the structure to be advanced along a plurality of paths. Each needle path first passes radially into and forwardly out of the end of the first body duct and into the hole of the second body duct. The path then everts so that the needles and associated sutures will pass outwardly through tissue peripheral to the hole when the end of the first body duct is on the structure adjacent to the hole in the second body duct. The needles preferably travel along such paths when they are advanced forward. In one embodiment, the device uses a J-shaped tube for guiding one of the needles along the desired path. In another embodiment, shape-memory needles having an arcuate profile are used to create the desired path.

U.S. Pat. No. 6,645,227 discloses a suture anchor including a placement portion frangably connected to an attachment portion. The attachment portion includes an outwardly projecting barb. During insertion of the suture anchor within a bore hole formed on a bone, the barb scores at least a portion of the bone bounding the bore hole. Once the suture anchor is disposed within the bore hole, a withdrawal force is applied to the suture anchor such that the attachment portion thereof rotates within the bore hole and disconnects from the placement portion. In an alternative embodiment, the suture anchor can be formed without the outwardly projecting barb.

U.S. Pat. No. 6,363,938 discloses an apparatus and methods provided for forming a channel in an organ or vessel to enhance perfusion therein. The apparatus may include a bioactive agent to stimulate tissue growth and vascularization in tissue adjacent to the channel. The apparatus includes a stent, suitable for percutaneous or intraoperative placement, that includes a tubular member having a plurality of tines, barbs, ribs or other structure to secure the stent at a desired position within tissue.

US 20050125020 discloses methods and apparatus for anchoring within the gastrointestinal tract. The present invention relates to an anchor configured for minimally-invasive implantation and sized to remain securely positioned within at least a portion of the gastrointestinal tract of an animal. The anchor includes a radial spring formed from an elongated resilient member shaped into an annular wave pattern about a central axis. The anchor defines a central lumen and provides an outward radial force, while allowing for substantial flexure about its perimeter. The anchor is generally removable, but can include fasteners, such as barbs, to further secure it to the surrounding anatomy. In some embodiments, the anchor includes a connector coupling a fixed portion to a removable portion. Further, the anchor can be used to secure a medical device within the body, such as a flexible sleeve within the intestine.

US 20040068217 discloses methods and apparatus for making an anastomotic connection between a first aperture in a side wall of a graft conduit and a second aperture in a side wall of a body tissue conduit using a hollow self-expanding exterior connector. The tissue about the first aperture is introduced into the hollow connector and is retained by first members of a distal portion of the connector. A delivery tool then collapses the distal perimeter defined by the first members by deforming the connector and delivers the first members into the lumen of the body tissue conduit via the second aperture. Upon inserting the first members into the body tissue conduit, the tool is disemployed and the connector reforms such that the first members and a more proximal portion of the connector external to the body tissue conduit press together the walls of the two conduits around the apertures.

WO 2005/004727 discloses a surgical fastening device for pinning a surgical filament to a body tissue. The device includes a grasping handle and a slender shaft extending from the grasping handle. A compartment contains one or more surgical fasteners. An ejecting mechanism is used to eject a surgical fastener from a compartment containing one or more surgical fasteners. The device also includes a filament dispensing system that dispenses surgical filament along the shaft so that a fastener grasps the filament when being ejected from the shaft. The invention also provides surgical fasteners and surgical filaments for use in the device.

WO 96/02211 discloses an intraluminal stent. An improved stent provides mechanical anchoring of the stent to a blood or other body vessel. The stent has, in a preferred embodiment, barbs (18) which remain within the surface of the stent when the stent is in its unexpanded condition, but which extend from the surface of the stent when the stent is expanded. These barbs (18) are adapted to engage, for example, a graft and/or the inner layers of a blood vessel to mechanically attach the stent to the vessel. Because friction is not solely relied upon to hold the stent in place, the stent may exert less force on the blood vessel which, in turn, means that a thinner stent requiring less force for expansion may be used. In addition, there may be less radial force permanently exerted in an artery after stent deployment which may be less injurious to the vessel.

WO 90/015582 discloses an aortic graft and system and method for implanting the aortic graft. The aortic graft comprises a substantially cylindrical graft material with attachment means which comprise a plurality of post and hook assemblies which are implanted with a double catheter system to penetrate the aorta wall above and below the aneurysm to provide firm attachment of the aortic graft and, thereby, exclude the aneurysm from the circulatory system.

WO 2003/099167 discloses an endoluminal device having barb assembly. An endoluminal device for implantation in a body lumen reduces movement or migration of the device after implantation by the use of barbs or barb assemblies. A first embodiment uses at least one barb assembly having first portions attached to an implant, a bend, and second portion disposed opposite the first portion from the bend and having a bearing surfaced. The second portion is adapted to protrude radially inward when the implant is in the radially compressed configuration and radially outward when the implant is in its radially expanded configuration. A second embodiment uses a barb having a curved segment which is curved proximally and radially inwardly. A third embodiment utilizes at least one barb assembly having a wire with a length greater than the cell height of the implant across which it extends and a substantially uniform cross-sectional area.

### SUMMARY OF THE INVENTION

The present invention relates to devices and methods of fastening objects to each other based on the penetration of at least one of the objects, which objects may thus each be made of fabric or the like natural or artificial material, which may but does not necessarily have to, be in the form of a layer of any shape including curvilinear or planar shapes, which further may be a tissue, including biological tissue such as, for example a wall of a blood vessel.

The term “fastening” in the context of this application, designates, in addition to the common meaning of this term, also connecting or fixating two or more objects one to each other, either touching each other or not, or to create a structure out of few objects in the form of layers connected together. If used in medical applications, such fastening can refer, for example, to anstomosis of blood vessels in various configurations (e.g., side to side, end to end, end to side), or to anchoring of objects such as fastening grafts, vascular bypasses, connecting artificial (aortic) valves to their place, anchoring a sensor within a cavity of human body, and the like.

According to one aspect of the present invention there is provided a fastening device to be associated with a first object, for the attachment of said object to a second object, the device comprising at least one fastening element having a base portion and at least one securing portion, said fastening element being originally produced with its securing portion having a first orientation relative to the base portion and with a possibility to bring the securing portion into a second orientation relative to the base portion, to allow said at least one securing portion to penetrate said second object being exposed thereto while staying in said second orientation, and being adapted to change said second orientation into said first orientation to provide said attachment.

The fastening element of the above device may comprise a restrainable portion between the base and securing portions, said fastening element being originally produced with its securing portion having said first orientation relative to the base portion and with the possibility to bring the securing portion into the second orientation relative to the base portion by restraining said restrainable portion so as to enable the securing portion to return from its second orientation to its first orientation when the restraint is released from the restrainable portion; the fastening element in said device having a penetration state in which said restrainable portion is restrained to keep said at least one securing portion in said second orientation constituting its a penetration orientation, to allow said at least one securing portion to penetrate said second object while staying in said penetration orientation, and being adapted to change said penetration state to an attachment state in which said restraint is released to let said at least one securing portion return to its said first orientation, constituting an attachment orientation thereof at which the first object is attached to the second object.

In the context of the present application, the term “restrainable portion” refers to a portion of the fastening element, whose state in this element as originally produced is considered herein to be an ‘unrestrained state’, and which may be deformed or mechanically operated when a so-called ‘restraining force’ is applied thereto directly or indirectly; the restraining portion returns to its original state when the restraining force is released.

According to another aspect of the invention, there is provided a fastening device with plurality of fastening elements as described above and a plurality of restraining elements, which may be in the form of holders mounted thereon, each for keeping the restraining portion of at least one fastening element in the restrained state, each of said restraining elements being adapted for applying thereto of an actuating force for releasing the restrainable portions and thereby letting the securing portions of the fastening elements to return into their attachment orientation.

It should be noted that the orientation of the securing portion of the fastening elements may be established not only with respect to the base portion but also with respect to an object to be penetrated or certain point or surface thereon.

The restraining elements may be movable relative to their fastening elements between a restraining position in which the restraining element applies, directly or indirectly, a restraining force to the restrainable portion to keep said at least one securing portion in said penetration orientation, and a releasing position to remove said restraining force from said restrainable portion, thereby bringing the at least one securing portion to said attachment orientation.

The fastening device may further comprise a carrier with at least one carrying portion to which more than one said fastening element is mounted. The carrier may be adapted for changing its state between a collapsed state in which the fastening elements are disposed relatively remote from said second object to be penetrated, and an expanded state in which at least a part of the fastening elements are brought into vicinity of the second object and are caused to penetrate said object. When the carrier is in its collapsed state, the fastening elements may be oriented in a direction different from the direction of penetration, the carrier being adapted to align the fastening elements with the direction of penetration prior to being brought into vicinity with the second object.

The fastening device may be produced with its carrier in the collapsed state as described above, in which state it may be kept by exerting thereon of a compression force thereon, e.g. by a sheath, with a possibility of essentially simultaneously aligning the fastening elements in the penetration direction, when the compression force is withdrawn. The expansion of the carrier and brining it into the collapsed state may be performed by the inflation and deflation, respectively, by a balloon.

The carrier may constitute a part of actuating means for applying actuating force to the restraining elements, which may be achieved by providing the carrier with an arrangement adapted to apply to each said restraining element said actuating force upon the carrier having expanded to a predetermined extent. Thus, for example, upon the penetration of an object to a predetermined extent, further expansion of the carrier may cause release of said restraining force, changing thereby the orientation of the securing portions of the fastening elements from the penetration orientation to the securing orientation.

Alternatively, each fastening element may be provided with an arrangement adapted to apply to the restraining element said actuating force upon said at least one securing portion having penetrated the second object to a predetermined extent. Thus, for example, upon the penetration of the second object until its contact with said holder, further expansion of the carrier may cause the holder to move relative to fastening element, thereby releasing said restraining force.

According to a further aspect of the present invention, there is provided a method for the attachment of a first object to a second object, comprising:
providing at least one fastening element extending in a penetration direction, projecting from or connected to said first object and having a securing portion, the fastening element being adapted to change its state between a penetration state in which the securing portion has a penetration orientation suitable to penetrate said second object, and an attachment state in which said securing portion has an attachment orientation transverse to the penetration orientation, wherein said attachment orientation of said securing portion is a predetermined state with which said fastening element was originally produced with a possibility to bring the securing portion into the penetration orientation by applying to the fastening element a restraining force and to enable the securing portion to return from its second orientation to said predetermined state when the restraining force is withdrawn;
causing at least the securing portion while in the penetration orientation, to penetrate said second object; and
withdrawing said restraining force to have the fastening element change its state to said attachment state.

Fastening devices according to the present invention may be advantageously used for the attachment to each other of tubular objects in a side-to-side, end-to-side or end-to-end manner. One example of such use is a side-to-side attachment of a stent graft deployed in a blood vessel to the blood vessel's wall. In such applications, the fastening device may constitute an integral part of a stent which is part of the stent graft, the stent constituting a carrier having at least one carrying portion on which said at least one fastening element is mounted. Alternatively, the fastening device may be formed separately from the stent graft, in which case said device further comprises a carrier with at least one carrying portion on which said at least one fastening element is mounted, the stent graft having at least one connection area, said carrier being insertable into the interior of the stent graft to allow said fastening element to penetrate said graft at, and to project from, said connection area. In this case, the fastening device needs to be deployed within stent graft. According to a still further aspect of the present invention, there are provided means and method for positioning and centering a fastening device relative to a blood vessel, by means of a stent having an upper and a lower portion, each being collapsible and expandable independently of the other, the fastening device being attached to the lower portion of the stent. In the original state of the stent, both portions are collapsed by an exertion thereon of a compression force, e.g. by a sheath. Further, the compression force is withdrawn from the upper portion of the stent, whose circumference is thus brought into contact with the surrounding blood vessel's wall, due to which a proper positioning and, possibly, centering of the lower portion of the stent and the fastening device attached thereto, is achieved relative to the blood vessel.

According to a still further aspect of the invention, there are provided means and method for centering said fastening device relative a stent graft or another tubular object within which it is to be deployed.

According to a still further aspect of the present invention, there is provided a stent adapted for being deployed in a blood vessel and having at least one connection area, the stent being adapted, at least after having been deployed in the blood vessel, to function as a fastening device and to comprise at least one fastening element such as those described above, projecting from said connection area in a penetration direction and having a securing portion capable of changing its orientation relative to said penetration direction between a penetration orientation allowing at least said securing portion to penetrate the blood vessel's wall, and an attachment orientation in which the securing portion is oriented transversely to the penetration direction and is adapted to at least partially press said vessel's wall towards said connection area of the stent.

The stent may have all features described above with respect to the carrier of the fastening device. In particular, the stent may be adapted for changing its state between a collapsed state in which the fastening elements are disposed relatively remote from the blood vessel's wall, and an expanded state in which at least a part of the fastening elements are brought into vicinity of the wall and are caused to penetrate the wall. When the carrier is in its collapsed state, said fastening elements may be oriented in a direction different from the direction of penetration, and wherein prior to bringing the fastening elements into the vicinity of the wall, the carrier is adapted to align the fastening elements with the direction of penetration.

The stent's carrier may have carrying portions to which said fastening elements are mounted, said carrying portions being adapted to change their shape and/or orientation relative to adjacent portions of the carrier, thereby changing special disposition of the fastening elements between their penetration disposition at which the securing portions thereof have said penetration orientation and said attachment orientation in which the securing portion is oriented transversely to the penetration direction. This may be achieved, for example, by said carrying portions being adapted undergo a plastic deformation for changing their shape and/or orientation, e.g. upon the carrier having been brought into said expanded state. Alternatively, the fastening elements may be capable of moving with respect to said carrier to change the orientation of said securing portion.

The stent's carrier may have a plurality of carrying portions and said fastening elements may be mounted to said carrying portion so as to be stationary relative thereto, the carrier constituting, or being connectable to, said first object, said carrying portions of the carrier being plastically deformable to change their initial state in which the fastening elements are directed in a penetration direction to penetrate said second object, to theirs final state in which the fastening elements are directed transversely to said penetration direction to at least partially press said second object towards said carrier.

According to a still further aspect of the invention there is provided a method for producing a fastening device with at least one fastening element as described above, to be associated with a first object, for the attachment of said object to a second object, the device comprising at least one fastening element having a base portion and a securing portion capable of changing its orientation relative to the base portion between a penetration orientation in which the securing portion is adapted to penetrate said second object, and an attachment orientation transverse to the penetration orientation, said method comprising:
producing said fastening element with said securing portion in an initial orientation at least similar to the attachment orientation with a possibility of changing said initial orientation to a penetration orientation when the restraining force is applied thereto; and
placing a restraining element on the fastening element so as to apply thereto said restraining force to bring the securing portion in said penetration orientation, said restraining element being movable in a direction along the fastening element, to remove said restraining force from the securing portion, thereby bringing the securing portion in said attachment orientation, the restraining element having an dimension in said direction shorter than the fastening element.

The fastening element(s) in the fastening device produced as described above may be so designed that their base and securing portions are separate parts mechanically connected to each other at said restrainable portion. Alternatively, some or all the fastening elements may be in the form of a single body elastically deformable at least at said restrainable portion. In this case, the fastening elements may comprises or be made of a super-elastic material or shape-memory material. The latter materials have the property of phase change under certain conditions, the fastening element being thus produced when the material is in its one phase (e.g. under high temperature), then is cooled to be transformed into its second state (e.g. at a lower temperature), to bring the securing portion of the fastening element into its penetration orientation, and later returns to its original state (e.g. by the temperature increase), when it needs to take its attachment orientation.

One specific state in which the fastening element as described above may be originally produced is a bent state achieved by applying to the fastening element bending force. Thus, said securing portion will be in its penetration orientation when the fastening element is in its non-bent state and will be in its attachment orientation when the fastening element is in its bent state. In this case, the fastening device may further comprise means for keeping the fastening element in its non-bent state by providing a compensating force against said bending force by at least one holder, and allowing the bending force to bend the fastening element at said bending area by moving of said at least one holder relative to the at least one fastening element.

According to a further aspect of the present invention, there is provided a fastening device for the attachment of a first object to a second object, the device comprising a carrier having at least one carrying portion and at least one fastening element mounted to said carrying portion so as to be stationary relative thereto, the carrier constituting, or being connectable to, said first object, said at least one carrying portion of the carrier being plastically deformable to change its initial state in which the fastening element is directed in a penetration direction to penetrate said second object, to its final state in which the fastening element is directed transversely to said penetration direction to at least partially press said second object towards said carrier.

### BRIEF DESCRIPTION OF THE DRAWINGS

In order to understand the invention and to see how it may be carried out in practice, embodiments will now be described, by way of non-limiting examples only, with reference to the accompanying drawings, in which:

FIGS. 1A and 1B are schematic views from above of a fastening device according to one embodiment of the present invention;

FIGS. 1C, 1D and 2 are the same views of the fastening device as shown in FIGS. 1A and 1B, respectively, in operation;

FIGS. 3A and 3B are schematic views from above of a fastening device according to another embodiment of the present invention, before and after its operation;

FIG. 4A to 4C illustrate different embodiments of fastening element which may be used in a fastening device according to the present invention;

FIGS. 5A and 5B are schematic views from above of a fastening device according to a further embodiment of the present invention, before and after its operation;

FIG. 5C is schematic view similar to that of FIG. 5B, of a fastening device according to an alternative embodiment of the present invention;

FIGS. 6A to 8B illustrate examples of different embodiments of fastening elements and their restraining means, which may be used in a fastening device according to the present invention;

FIGS. 9A to 11 illustrate examples of different dispositions of fastening elements with respect to a carrier, which may be used in a fastening device according to the present invention;

FIG. 12 illustrates examples of manners of operation of a fastening element according to different examples of the present invention;

FIGS. 13A to 13B illustrate examples of designs of different portions of a fastening element which may be used in a fastening device according to the present invention;

FIGS. 14A to 15 illustrate examples of various cross-sectional shapes of securing portions of fastening elements and corresponding holders, which may be used in a fastening device according to the present invention;

FIGS. 16A to 16C illustrate a fastening element and its operation in a fastening device according to a further embodiment according to the present invention;

FIGS. 17A to 18C illustrate examples of various designs of a carrier which may be used in a fastening device according to the present invention;

FIGS. 19A and 19B illustrate further alternative designs of fastening elements which may be used in a fastening device according to the present invention;

FIGS. 20A to 21B illustrate examples of positioning means which may be used to position a fastening device according to the present invention;

FIG. 22 illustrates a fastening device similar to the one shown in FIG. 1C, in a centered position in a blood vessel;

FIGS. 23A to 23C illustrate one example of a balloon which may be used with a carrier of the kind shown in FIGS. 17A to 17E;

FIGS. 24A to 26F illustrate different examples of the process of deployment of a fastening device of the present invention, into a blood vessel;

FIG. 27 illustrates an arrangement of fastening elements in a fastening device constituting a part of a stent, according to a further embodiment of the present invention; and

FIGS. 28A to 28C illustrate different kinds of attachment of objects one to another by means of fastening devices according to the present invention.

### DETAILED DESCRIPTION OF EMBODIMENTS

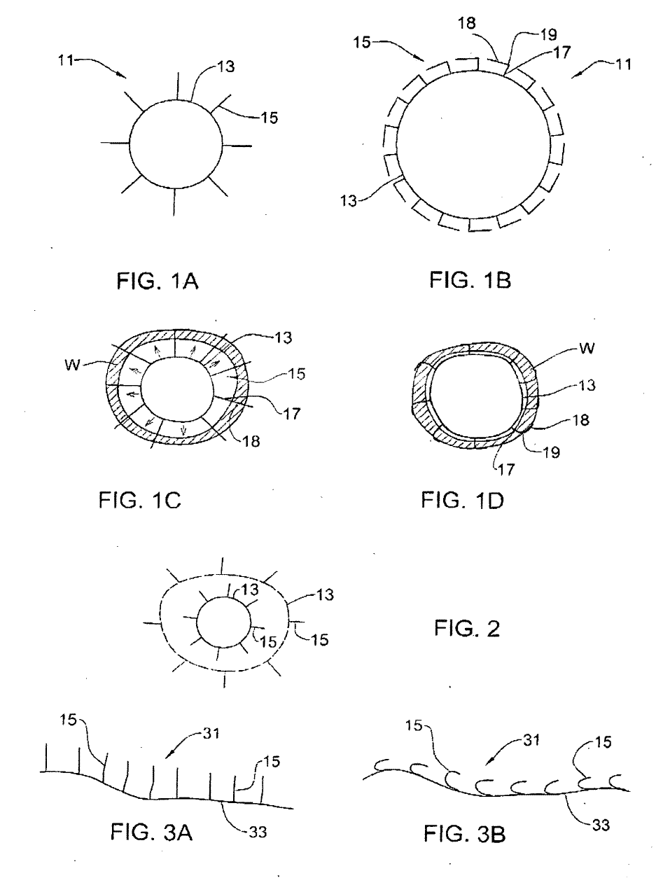

One, simplified example of a fastening device according to the present invention is shown schematically in FIGS. 1A and 1B. The fastening device 11 comprises a carrier 13 and a plurality of fastening elements 15 attached thereto. As shown in FIG. 1B, each of the fastening elements 15 comprises a base portion 17, a securing portion 18 and a restrainable portion 19 therebetween, and is adapted to take different states in accordance with different orientations of its securing portion 18, the states being a penetration state as shown in FIG. 1A and an attachment state as shown in FIG. 1B.

Generally, the fastening device 11 can constitute a part of a first object to be attached to a second object by the fastening elements penetrating the second object, or it may be used for the attachment of one object to one or more other objects by the penetration of all these objects by the fastening elements. In the former case, in FIGS. 1A and 1B, the carrier 13 of the fastening device 11 may for example constitute a part an object having a circular outer surface, which is to be inserted within a tubular object and attached to its inner surface by fastening elements' penetration therein. In the latter case, the fastening device 11 may be used for example for fastening a first tubular object to a second tubular object surrounding it, in which case the device will be deployed in the first object, and the two objects will be fastened together by the fastening elements' penetration of their walls.

A more particular example of the use of the fastening device 11 for the attachment of a stent graft aimed to be deployed in and attached to a blood vessel by the penetration of the blood vessel's wall by the fastening elements 15 and securing the attachment thereby. The fastening device can constitute an integral part of the stent, in which case the carrier 13 will be a carrying portion of the stent, i.e. its portion at which the fastening elements 15 are mounted and from which they protrude to penetrate the blood vessel's wall. The fastening device can also be produced separately from the stent and may be adapted for being deployed therein. In this case, the stent graft will have a connection area (not shown), and the fastening elements will penetrate the graft in said area and protrude outwardly therefrom. In the fastening device FIGS. 1A and 1B the connection area may be imagined as being disposed adjacent outer surface of the carrier 13, and being penetrated by the fastening elements 15 to further penetrate a blood vessel's wall.

In view of the above, and for better understanding only, in further description of the fastening device 11 and other fastening devices having carriers, each carrier should be considered, where appropriate, to constitute either the carrying portion of a stent with which it is integrally formed, or the connection area of a stent graft into which it is deployed.

FIGS. 1C and 1D illustrate the process of the attachment of the fastening device 11 to a blood vessel's wall W. As seen FIG. 1C, during the penetration of the fastening elements 15 into the wall W, the securing portion 18 thereof is generally aligned with the base portion 17 in a penetration direction shown by arrows. As seen in FIG. 1D, upon the completion of the penetration, the securing portion 18 is brought into its attachment orientation in which it is transverse to the base portion 17 to press the wall W against the carrier 13.

It should be noted that though in FIGS. 1A to 1D the penetration direction is shown generally radially with respect to the carrier 13, this does not necessarily have to be the case. Thus, alternatively, the penetration direction may be parallel to a longitudinal axis of the carrier as in the case of e.g., end to side or end to end anastomosis, or may have any other direction depending on the geometry and disposition of objects to be fastened by the fastening device.

The above is achieved by producing fastening element 15 from an appropriate material as described below, so that, in an initial state, its securing portion 18 has an orientation similar or identical to its planned attachment orientation, then bringing the securing portion 18 into the penetration orientation and restraining the restrainable portion 19 to keep the securing portion in this orientation during the entire penetration process (as will be described in more detail below), and finally releasing the restrainable portion 19 to bring the securing portion 18 to the attachment orientation. The securing portion 18 in its attachment orientation does not have to be strictly perpendicular to the base portion 19 but rather may form therewith any obtuse or acute angle, and in its penetration orientation it does not have to be fully aligned with the base portion 19, but may rather be slightly inclined relative thereto.

The fastening elements may be made of an elastic or super elastic material, shape memory material or alloy, e.g. such as Nitinol or any other appropriate material allowing the fastening elements 15 to be first elastically deformed, e.g. at their restrainable portion, then kept in the deformed state for a period of time by a restraining means acting on the latter portion, and finally be returned to their initial, deformed state by releasing the restraining means. The simplest example of the above deformation of the fastening element 15 is its bending at the restrainable portion 19.

Referring to FIG. 2, the carrier 13 may be adapted to change its state between a collapsed or shrunk state shown in solid lines and an expanded state shown in dotted lines. It should be noted that fastening elements 15 shown during collapsed state do not have to necessarily extend in penetration direction as shown in this figure, and as will be explained in more detail below, measures may be taken to direct them in the penetration direction while the device is still in its collapsed state. This may be particularly useful for medical applications where the fastening device needs to be first introduced into a lumen or a cavity such as a blood vessel, and then expanded to let the fastening elements 15 approach the cavity's wall and actuate their penetration into it. The carrier 13 can be expanded by any suitable means such as the inflation of a balloon introduced therein (not shown). Alternatively, the carrier 13 can be self-expandable, in which case it is adapted to be originally held in the collapsed state, directly or indirectly, by a sheath (not shown), the expansion being achieved by the removal of the sheath. The carrier 13 does not necessarily need to have a closed or circular shape but rather it may have any spatial shape, depending on the objects to be fastened. FIGS. 3A and 3B, for example, show a fastening device 31 having a carrier 33 in the form of an elongated, open structure with fastening elements 15 in their penetration state (FIG. 3A) and attachment state (FIG. 3B). The carrier 13 may be made of any appropriate material allowing it to behave as described above. The material may have high elasticity and it may be different from, or of the same kind as, that of which the fastening elements 15 are made.

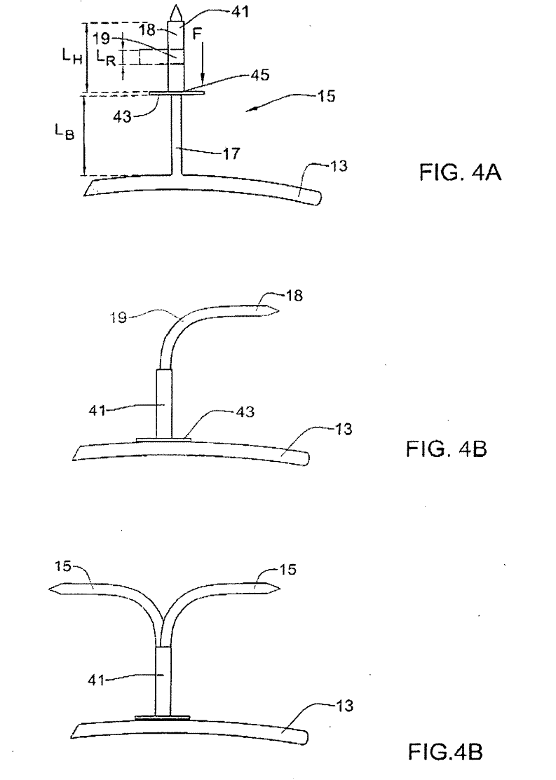

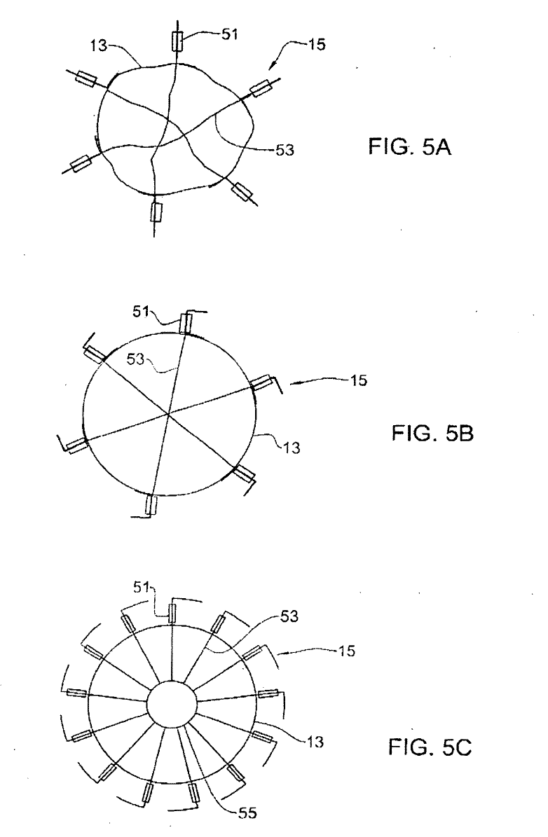

With reference to FIGS. 4A, 4B, and 5A to 5C, examples of restraining means will now be described, used with each fastening element 15 to keep its securing portion 18 in the penetration orientation during the penetration process as described above and shown in FIG. 1C. In all these examples, the restraining means is in the form of a holder 41 (FIGS. 4A and 4B) or 51 (FIGS. 5A to 5C), which is a sleeve or a cannula movably mounted on the fastening element 15. The holder 41, 51 has a length L_{H} (designated only in FIG. 4A) which depends on the length L_{R} of the restrainable portion 19 and the length L_{B} of the base portion 17 of the fastening element 15 as follows: L_{R}≦L_{H}≦L_{B}. The length L_{H} of the holder is such as to cover at least a part of the restrainable portion 19 of the fastening element 15 and to leave uncovered at least a part of the base portion 17 adjacent the carrier 13, to allow the movement of the holder along the fastening element 15 in the direction towards the carrier 13 to the extent necessary to release the restrainable portion 19. The fastening element 15 may also have means preventing the movement of the holder 41 in the direction away from the carrier 13, to avoid its detachment from the fastening element 15. Holders such as those described above may be adapted to hold more than one fastening element, as shown in FIG. 4C.

The holder 41, 51 is adapted to move when an actuating force F is applied thereto during the penetration process, which is achieved in different ways as shown in the respective figures as explained below.

In the holder 41 in FIGS. 4A, 4B and 4C, this movement is achieved by means of providing the holder with a flange 43 disposed at a proximal end 45 thereof and laterally projecting therefrom, and the actuating force F is applied thereto during the penetration, by an object being penetrated such as a blood vessel wall or a tissue (not shown). As shown in FIG. 4B, once the holder 41 has moved by the actuating force to an extent that it at least partly uncovered the restrainable portion 19 of the fastening element 15 and, consequently, the restrainable portion 19 is released (by returning to its original, non-restrained state) and, accordingly, the securing portion 18 changes its orientation to the attachment orientation.

In the holder 51 in FIGS. 5A to 5C, the actuating force is applied to the holder by means of a cable 53 connected thereto at its one end. The other end of the cable 53 is adapted to actuate the cable by bringing it from its loosened state (FIG. 5A) to its tightened state (FIGS. 5B and 5C), to cause the holder to move relative to the fastening element 15. The tightening of the cable 53 may be achieved by any appropriate means, either remotedly or for example by arrangements connected with the change of the state of a carrier 13 on which the fastening element are mounted. This carrier may be such as described above with reference to FIG. 2, and the change of its state will thus be between a collapsed state (FIG. 5A), in which the cable is loosened, and an expanded state (FIG. 5B, 5C) in which the cable 53 is tightened. In the example of FIGS. 5A and 5B, the cable 53 of one fastening element 15 is connected to the holder 51 of a fastening element on an opposite side of the carrier 13. When as shown in FIG. 5B, the carrier 13 is brought to its expanded state, in which it has a predetermined final size, e.g. by any of the means mentioned above, the cable 53 whose final length is also predetermined, pools the holders 51 towards each other and, consequently, towards the carrier 13, thus causing them to move towards the carrier at least until they are sufficiently removed from the restrainable portions 18 of the corresponding fastening elements 15 and these portions are, consequently, released. As a result, the securing portions 17 change their orientation from the penetration orientation to the attachment orientation and the fastening elements 15 change their state from the penetration state to the attachment state which is identical to their initial state. In the example of FIG. 5C, the holders 51 of all the fastening elements 15 are connected to an element within the carrier, such as for example a central ring or circular wire 55. Alternatively, in the example of FIG. 5C, all the holders 51 may be connected by a single cable extended along the circumference of the carrier 13 (not shown), The final outcome in the latter case, as well as in the case of FIG. 5C is similar to that presented in FIG. 5B, where the fastening elements 15 change their state to the attachment state due to the movement of the holders 51.

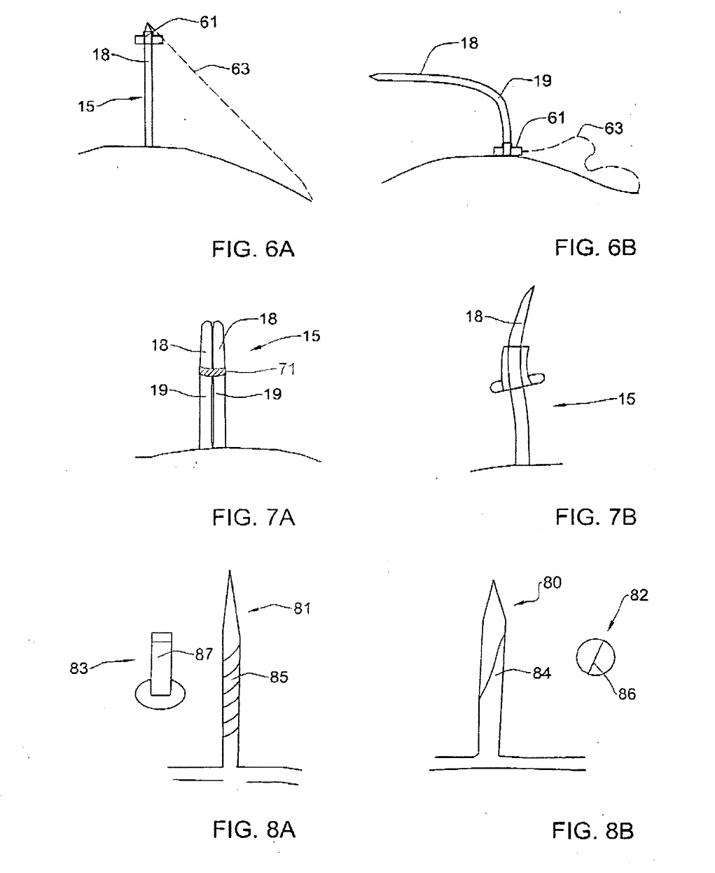

FIGS. 6A and 6B show an alternative example of a restraining means in the form of a holder 61 mounted solely on the securing portion 18 of the fastening element 15 (without covering the restrainable portion) and movable therealong when if an actuating force is applied thereto by an object under penetration. The restraining means in this example further include a cable 63, one end of which is connected to the holder 61 and the other end is controlled to be tightened (FIG. 6A), from a direction opposite to the direction in which the securing portion 18 has to move to take its attachment orientation (FIG. 6B). The tightening force needs to be applied to the cable at least as long as the holder 61 is located on the securing portion 18 or on the retainable portion 19.

It should be noted that the holder 41, 51 and 61 does not need to be in the form of a sleeve but may have any other form suitable or not for the holder to enter an object to be penetrated together with the securing portion 18 of the fastening element 15. Moreover, it may be shorter than the length of the restrainable portion or may not cover it at all. For example, as shown in FIG. 7A the holder may be in the form of a stopper 71 having, e.g., a flat surface, similar in its dimensions and operation to the flange 43 of the holder 41. The stopper 71 may be particularly advantageous for use with two fastening elements, as shown, with their securing portions 18, tending to bend in opposite directions at their restrainable portions 19. The stopper 71 thus holds them together between their tips and the restrainable portions.

In addition, the above described holders may be of a design allowing to keep the fastening element 15 with its securing portion 18 at a desired inclination relative to the base portion, as shown in FIG. 7B. In the latter example, the inclination may be in the direction opposite to the direction in which the securing portion 18 tends to move to take its attachment orientation.

In all the above examples, the holders are kept in their initial position on the fastening elements 15 by friction, being tightly mounted thereon, and when caused to move along the fastening elements by an actuating force, this force has to be greater than the friction force. However, as will be described below with reference to FIGS. 14A to 14C, to achieve this, the holders do not need to contact the fastening elements along their entire circumference or entire length.

In addition, to facilitate the interaction between holders and the fastening elements, their interacting surfaces may have specific mating shapes to provide a rotational component to the movement of the holders along the fastening elements. FIGS. 8A and 8B show different examples of such arrangements. In FIG. 8A, a fastening element 81 has a threaded portion 85 and a holder 83 has a corresponding screwed portion 87 adapted to interact with the threaded portion and thereby cause the holder, when an actuating force is applied thereto, to move in a spiral manner along the fastening element 81. In FIG. 8B, a fastening element 80 has an inclined slot 84 and a holder 82 has a corresponding projection 86 adapted to be received within the slot 84 and thereby cause the holder when an actuating force is applied thereto, to move in a spiral or the like manner along the fastening element 80.

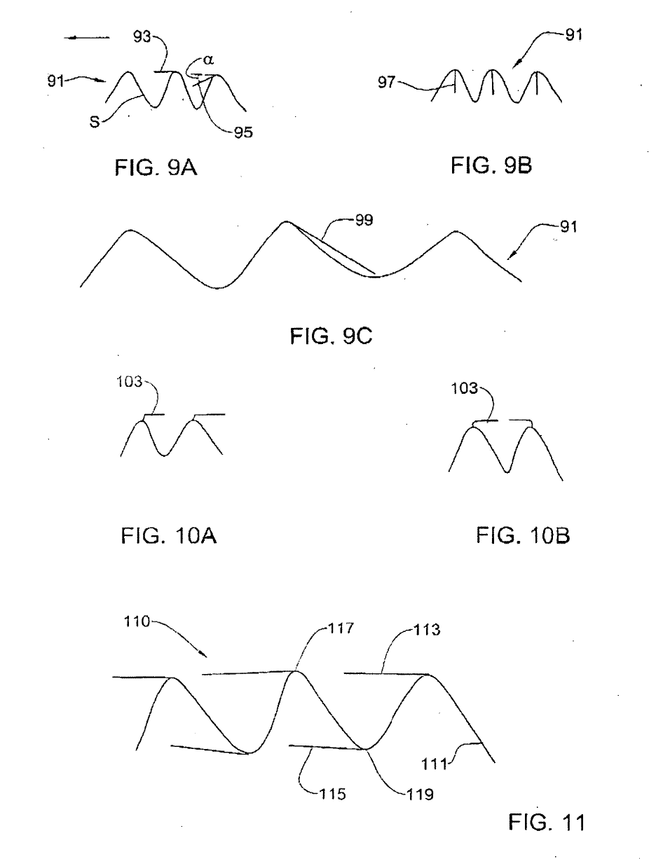

Fastening elements, in their penetration state may have initial spatial disposition with respect to a carrier, different from that in which the fastening element is directed in the direction of penetration. In these cases, measures need to be taken to move the fastening elements from their initial disposition to alignment with the direction of penetration. Some examples are shown in FIG. 9A to 9C, of such different dispositions of fastening elements with respect to a carrier which is in the form of a z-stent 91 having struts S. The struts S have their height parallel to the stent's central axis. In FIG. 9A a fastening element 93 is oriented generally along a circumferential direction of the stent 91, which is perpendicular to the height of the struts S and is designated in FIG. 9A by an arrow, and in a fastening element 95 is titled with respect to the circumferential direction. FIG. 9B shows fastening elements 97 perpendicular to the circumferential direction of the stent. FIG. 9C shows fastening elements 99 oriented along a part of the stent's struts. As shown in FIGS. 10A and 10B fastening elements 103, when in their penetration state, may have different dispositions in with respect to each other.

A fastening device according to the present invention may comprise more than one array of fastening elements arranged at different locations along its height. FIG. 11 shows a fastening device 110 with a carrier in the form of a z-stent 111, and a double array of fastening elements 113 and 115 in their attachment state, which are mounted to upper and lower extremities 117 and 119 of the stent's struts. The location of the fastening elements 113 and 115 on the carrier 111 is not limited to the strut's extremities, but can be at any suitable points along the struts. In addition, the carrier may have struts of different heights on which fastening elements can be fixed as well.

Various embodiments of a fastening element according to the present invention will be now discussed, in a fastening device with any restraining means described above.

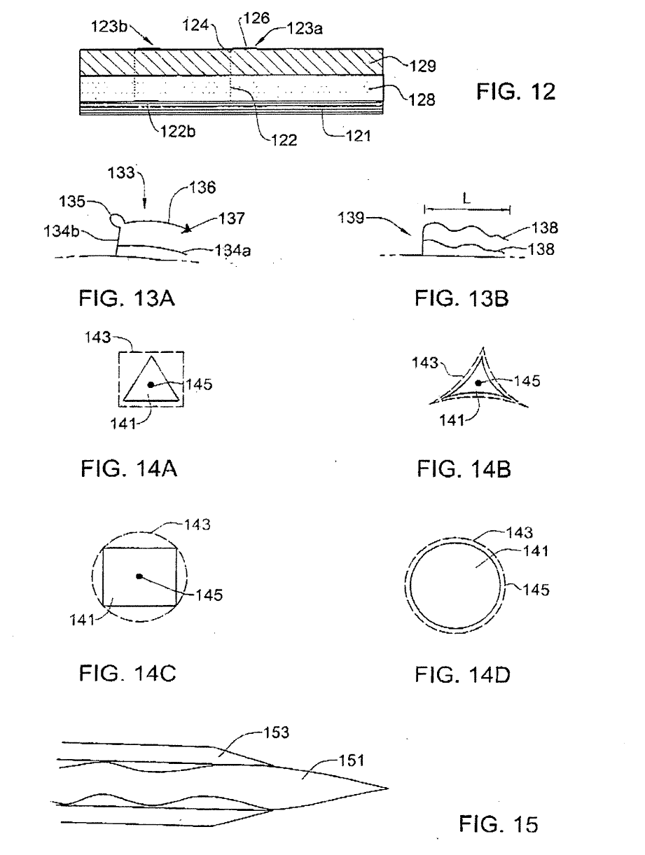

The fastening element can be either a single body having the base, the restrainable and the securing portions as described above or it can have these portions mechanically connected to each other. In both options, the restrainable portion 19 of the fastening element is adapted to be restrained as described above, and the securing portion 18 is adapted to change its orientation relative to the base portion 17 due to the release of the restrainable portion 19. FIG. 12 presents examples of two types of fastening elements 123a and 123b mounted to a carrier 121, for the fastening of two objects 128 and 129. The fastening element 123a, being either a single body or being in the form of an assembly, comprises a base portion 122 (shown in dotted line), a restrainable portion 124 and a securing portion 126. In the penetration state of the fastening element 123a (not shown), the base portion 122, the restrainable portion 124 and the securing portion 126 penetrate both objects 128 and 129. Then the restrainable portion 124 is released and the securing portion 126 changes its penetrating orientation to the attachment orientation, wherein the objects 128 and 129 are pressed against the carrier 121. The fastening element 123b has an additional base portion 122b connected to the carrier 121. This additional portion allows the objects 128 and 129 to be pressed against the additional portion 122b and not against the carrier 121. Therefore, in the absence of a carrier, a fastening element 123b can function both as a carrier and a part of a fastening element.

The fastening element may have more than one of each of the previously mentioned portions, each of which do not necessarily have to be straight as described in previous examples. FIGS. 13A and 13B present examples of various designs of different portions of the fastening element. FIG. 13A shows a fastening device, comprising a fastening element 133 having two base portions 134a and 134b, a restrainable portion 135 and a securing portion 136. The base portion 134a is essentially parallel to the securing portion 136 so that to allow the securing portion 136 when in the attachment orientation as shown, to press an object to be penetrated thereby towards the base portion 134a. The securing portion 136 has an arrow-like tip 137 at its top, to facilitate its penetration through the objects to be fastened. It should be noted that securing portions described in this application may have any appropriate form to allow penetration into an object, e.g. it may be in the form of a barb or a hook.

The restrainable portion 135 is shown in FIG. 13A as having an open circular shape to allow more parallelism between the securing portion 136 when in the attachment orientation, and to the base portion 134a, as well as small radius bending of the restrainable portion 135. FIG. 13B shows a fastening element 139 having two securing portions 138 of a sinus-like shape, which can be any other periodic or non-periodic shape, wherein the projected length L of the securing portion is shorter than its actual length. This kind of the securing portions may provide better tightening of the objects to be fastened.

Referring to FIGS. 14A-D, some examples are presented of the various shapes of the cross-sections of a securing portion and a corresponding holder that may be used in any fastening element described above. These shapes may be chosen to facilitate the penetration of the fastening element into object(s) to be penetrated, due to the reduction of the total area of contact therebetween and, consequently, the friction force to be overcome by actuating force applied to the holder. The securing portion 141, whose different possible cross-sectional shapes are shown in FIGS. 14A, 14B and 14C, have a distal end and a proximal end (not shown) and the shown cross-sections refer mostly to their proximal end which at their distal end they may have a sharp tip 145. FIGS. 14A-14B further show (in a dotted line) the cross-sectional shape of a holder 143, when mounted on the corresponding securing portion, to hold it in the penetration orientation. The securing portion 141 can be in full contact with its holder 143, along the whole circumference of its cross section, as shown in FIG. 14B, or it may contact the securing portion 141 only partially, as shown in FIG. 14A, or at selected contact points as shown in FIG. 14C. FIG. 14D shows a circular securing portion 141 and its holder 143. The securing portion and the holder 143 do not necessarily have to contact all along their circumferences. The cross section of the securing portion 141 may be smaller than that of the holder 143. In the latter case, as well as all other described examples, wherein a certain gap exists between the securing portion 141 and the holder 143, the gap can be filled with a material having a viscosity sufficient to provide frictional interaction therebetween. Another example of interaction between a securing portion and a holder is shown in FIG. 15. Due to the special shape of the securing portion 151 and the holder 153 mounted thereon, the total area of contact therebetween is reduced.

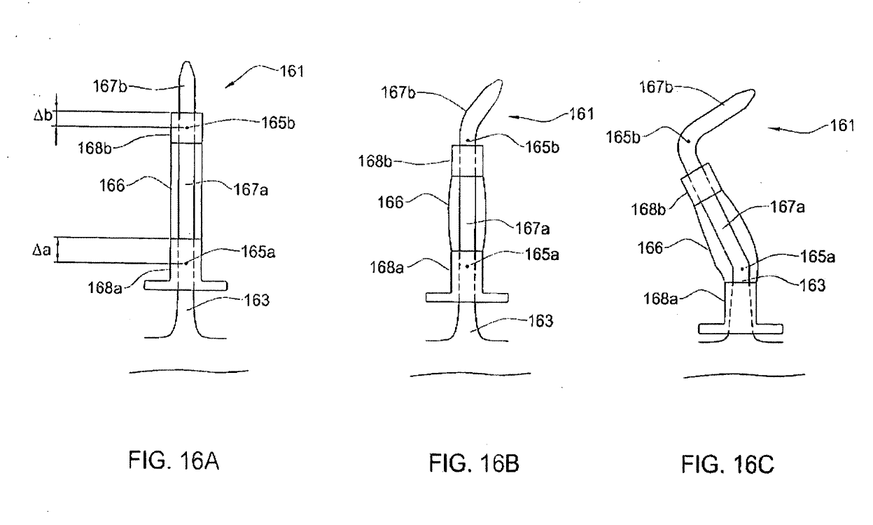

The fastening element according to the present invention can have more than one couples each comprising one restrainable portion and one securing portion. FIGS. 16A to 16C show a fastening element 161 comprising a base portion 163, a restrainable portion 165a, a securing portion 167a, a further restrainable portion 165b and further securing portion 167b. The fastening element further comprises two holders 168a and 168b, for keeping each restrainable portion 165a and 165b restrained. These portions may be connected by wires 166, to allow a mutually coordinated movement thereof. In particular, the holder 168a is longer than the holder 168b, and together with the wires 166 they are designed and mounted in such a way that during penetration, downward movement of the holders 168a and 168b, will cause restrainable portion 165b to be released first. This can be shown by the following relation: the distance Δa between of the upper edge of holder 168a to restrainable portion 165a is longer than the distance Δb between of the upper edge of holder 168b to restrainable portion 165b. In operation, first, the fastening element 161 is in its penetration state (FIG. 16A), then during the penetration, the holder 168a moves down, pulling the holder 168b by cables 166, due to which the restrainable portion 165b is released whereas restrainable portion 165a is not. Consequently, only the securing portion 167b changes its penetration orientation to its attachment orientation (FIG. 16B), whereas the securing portion 167a is still in its penetration orientation. While the penetration continues, the holder 168a continues to move down, until the holder 168a sufficiently uncovers the restrainable portion 165a, which is thereby released. Consequently, the securing portion 167a changes its penetration orientation to its attachment orientation as shown in FIG. 16C.

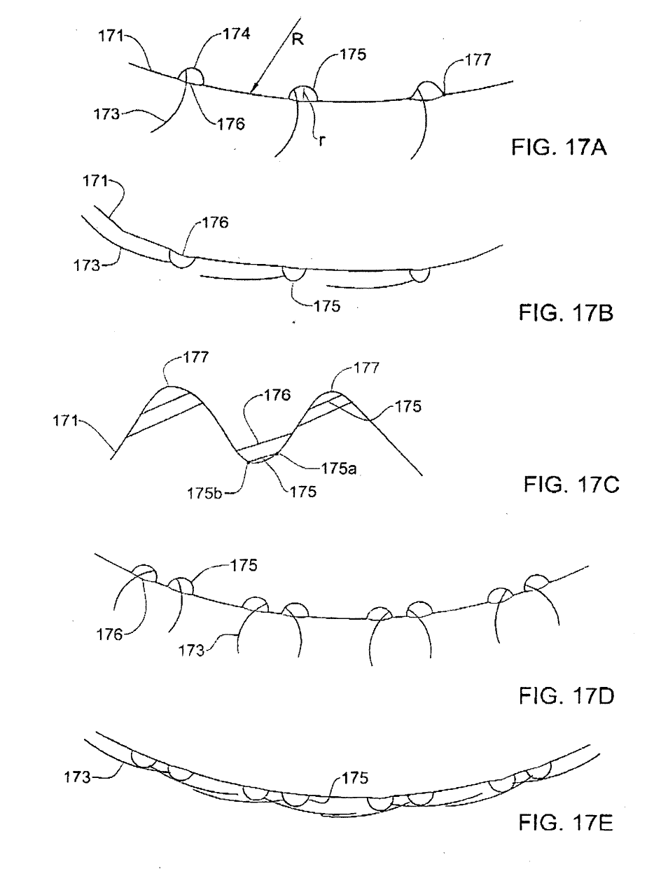

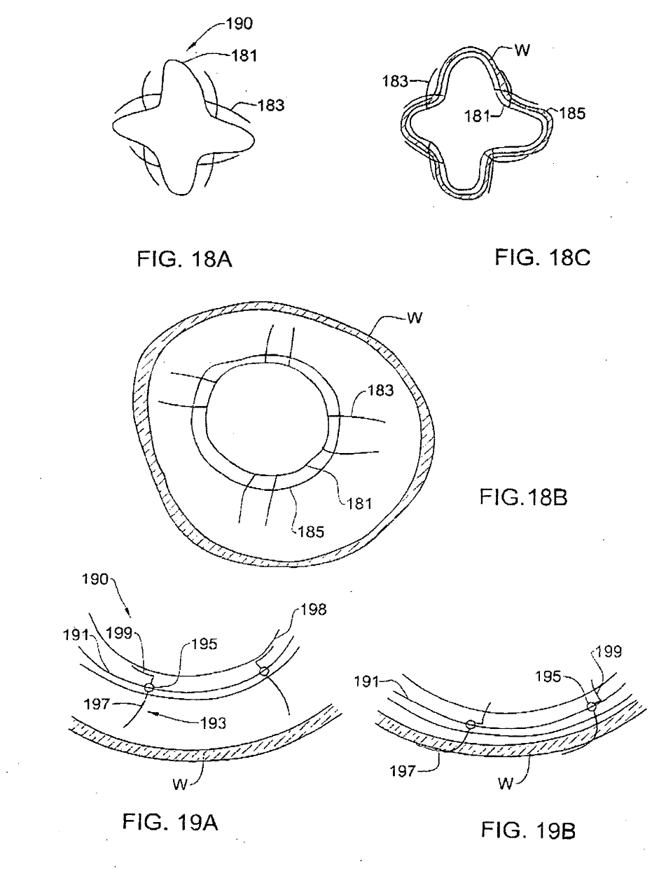

FIGS. 17A to 18C present fastening elements according to the present invention whose states change due to the change in the form at least a portion of a carrier these elements are connected to.

FIGS. 17A and 17B show a portion of a fastening device with a carrier 171 comprising a plurality of arc-like carrying portions 175 to each of which a fastening element 173 is attached at an area spaced from its apex 174. The carrying portions 175 have their radii r essentially smaller than radius R of the carrier 171, and have connections 175a and 175b at which the carrying portions merge with the remainder of the carrier. The carrying portions are so designed that, when a radial force is exerted thereon from the inside of the carrier 171, e.g. by a balloon, they bend to change their form from a concave to a convex one. In consequence with this, the fastening element 173 changes its state from the penetration state (FIG. 17A) to the attachment state (FIG. 17B). It is to be noted that during this change, the fastening element 173 does not change its form. The change in the form of the carrying portion 175 may be achieved while the carrying portion 175 is in the plastic region of a material, from which the carrier 171 and the carrying portion 175 are made. Such a material may be stainless steel or any other suitable material. As seen in FIG. 17C, the fastening device is an integral part of a stent, whose struts constitute the carrier 171 and have limiting struts 176 adjacent bending points 177 of the stent which are stronger than the bending points 177 (e.g., thicker width than the struts of carrier 171). The struts 176 are designed to limit the bending of bending points 177 of the carrier 171. Consequently, when a balloon (not shown) is inflated within carrier 171, it results in the expansion of carrier 171 till the struts 176 limits any further expansion. The design of the struts 176 is predetermined based on the maximal diameter to which the carrier 171 should expand during penetration. The expansion force necessary for this is lower than that required to change the form of the carrying portions, i.e. to bend it at the bending points 177, whereby during the expansion of the carrier the carrying portions are not deformed. When the force reaches the value required for the carrying portions to deform, with the struts 176 limiting any further expansion of the carrier 171, the force (i.e. additional inflation energy of the balloon) is transferred to the carrying portions 175 and deform them, thus bringing the fastening elements 173 from their penetration state to attachment state.

FIGS. 17D and 17E show a carrier 171 with another configuration of the carrying portions 175 and the fastening elements 173 connected thereto, in their penetration state (FIG. 17C) and the attachment state, wherein the overlapping of the fastening elements 173 can improve the attachment.

FIG. 18A shows an embodiment of an anastomosis fastening device 190 to be deployed within a stent graft 185 for attaching the stent graft) to a blood vessel's wall W in a side-to-side manner the attachment process being shown in FIGS. 18B and 18C. The fastening device comprises a carrier 181 of an essentially lobed shape and fastening elements 183 connected thereto in their initial (collapsed) state (FIG. 18A). The fastening elements 183 are arranged in a crisscrossing couples which, when in their collapsed state, may cover most of the surface of the lobes of the carrier 181, being essentially parallel thereto. The fastening elements are so mounted on the carrier 181 as to be capable to change their initial state to a penetration state (FIG. 18B) and then back to the attachment (collapsed) state similar to their initial state (FIG. 18C,), in which the carrier 181 tightly attaches the stent graft 185 to the vessel wall W. The expansion of the carrier 181 from the collapsed state (FIG. 18A) to the expanded state (FIG. 18B), and its collapsing back to the collapsed state (FIG. 18C) is achieved by means such as inflation and deflation of a balloon (not shown).

FIGS. 19A and 19B show another embodiment of a fastening device 190 according to the present invention, to be attached to a blood vessel wall W, comprising a carrier 191, which may be a stent graft, fastening elements 193 movably attached thereto and rotating means 195, which allow the fastening elements 193 to rotate with respect to the carrier 191. The rotating of each fastening element 193 may be limited to a desired direction and to a specific angle of rotation by means such as a spring (not shown) fixed to the carrier 191. The fastening elements 193 have a securing portion 197 and a base portion 199, which both change their orientation from a penetration orientation, as shown in FIG. 19A, to an attachment orientation, as shown in FIG. 19B, due to the rotation of the rotating means 195. The fastening element 193 can be brought to the penetration state by the inflation of a balloon 198 which will press the base portion 199 thereby preventing the rotation means 195 from rotating. As far as the balloon is inflated, the carrier 191 expands, and the fastening elements 193 can penetrate the wall W. A deflation of the balloon afterwards will release the rotating means and will allow the fastening element 193 to be rotated to the attachment state. It is to be noted that during these changes, the fastening element 193 does not change it form.

The process of using a fastening device according to the present invention will now be explained with respect to its use for the treatment of abdominal aortic aneurysms, the underlying weakness in the wall of the aorta which may rupture if it is too large. In the treatment procedure a stent graft is inserted into the aorta (hereinafter: ‘blood vessel’) and then expanded to be fixed therein. The stent graft is aimed at excluding the flow through the aneurysm with subsequent restoration of normal blood flow (via the graft). This, in turn, prevents the aneurysm from rupturing. Any of the fastening devices described above may thus be used for the fixation of the stent graft to the wall of the aorta, rather than, or in addition to, fixating stents by a radial force exerted thereby, as in the known stent-grafts. This is specifically advantageous in cases where the aneurysm has approached significant limb or artery such as the renal arteries. In such cases there is no healthy aorta wall available in sufficient length (no ‘neck’) for the deployment of a commercially available stent-graft based on radial force fixation.

In general, in order to use a fastening device according to the present invention, for the above purpose the following stages need to be performed:
1. Preparation of the fastening device for the insertion into the blood vessel; since as described above the fastening device may be produced integrally with a stent graft or separately therefrom, in further description any reference to the carrier of the fastening device should be understood respectively as a stent graft to which fastening elements of the fastening device are mounted, or a connecting area of a stent graft penetrated by fastening elements of a fastening device deployed therein;
2. Delivery to the site and positioning the fastening device inside the blood vessel together with or subsequently to the insertion of the stent graft;
3. Bringing the fastening device to a state ready for penetration of the blood vessel;
4. Penetration process;
5. Attachment of the stent graft to the blood vessel.

Stage 1 above is usually a part of manufacturing procedure, whereas stages 2 to 5 above are conducted within a patient and together are referred to as a deployment process.

Preparation of the Fastening Device for the Insertion into the Blood Vessel

When inserted into the blood vessel the fastening device should be in a folded or collapsed state, in order to be small enough to be delivered through the relevant parts of the vascular system. In such state the carrier is in its collapsed state and the fastening elements are in their penetration state, but in a disposition different from the desired deposition they should take for the securing orientation of their securing portions to be in the direction of penetration. Examples of such collapsed dispositions were previously described with reference to FIGS. 9A to 10B. In order to keep the fastening elements in these dispositions an external sheath or other suitable means can be used. In addition to the fastening elements, the same sheath can also hold the carrier in its collapsed state, when the carrier is of a self-expandable type.

### Delivering to Site and Positioning the Fastening Device Inside the Blood Vessel

The fastening device is delivered to the aneurysm according to common methods known in the art with the use of e.g., guide wires and catheters. Once the folded fastening device has been delivered to the aneurysm, the positioning takes place. In order to position the fastening device in the correct location inside the blood vessel positioning means are used.

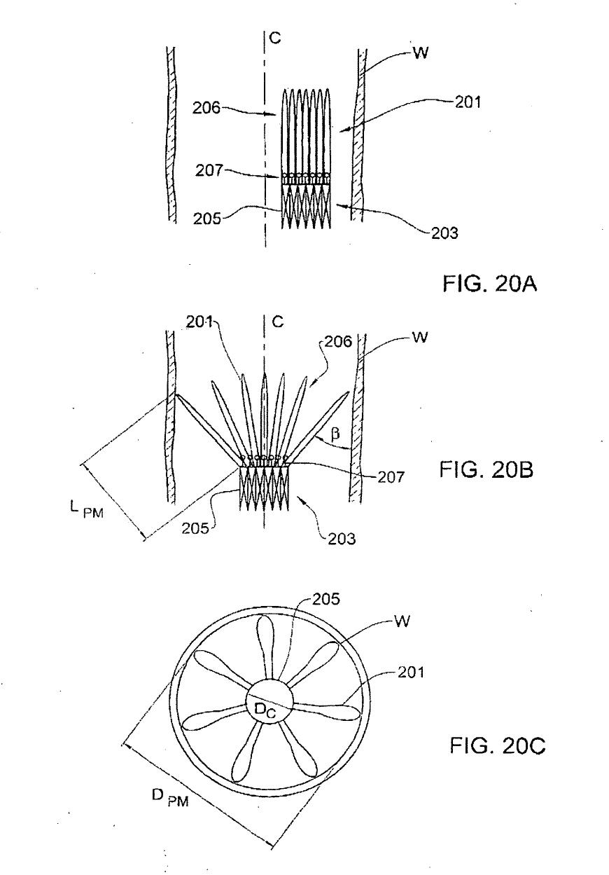

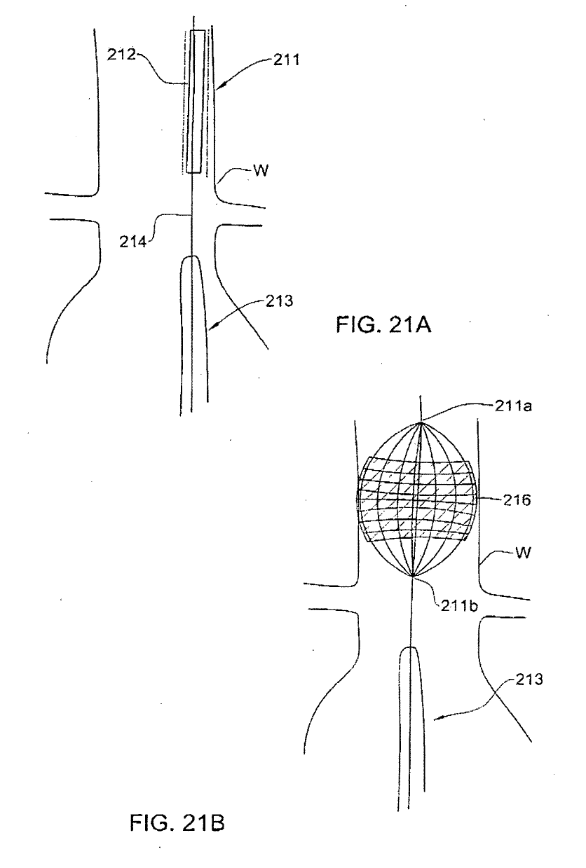

With reference to FIGS. 20A to 21B some examples of such positioning means will now be described. FIGS. 20A to 20C show a positioning means 201 in the form of a long-profile positioning stent, having an upper portion 206 and a lower portion 207 which constitutes a carrier 205 of a fastening device 203. The positioning means 201 may be formed integrally with the fastening device 203 and may or may not be a part of a stent graft, or, alternatively, the fastening device can constitute a separate device connected directly or indirectly (by means such as cable) to the positioning means, as will be further shown in FIGS. 21A and 21B. The positioning means 201 with the fastening device 203 is delivered to the interior of the blood vessel as shown in FIG. 20A, with both the means 201 and the device 203 being in their collapsed state, e.g. being covered by a sheath (not shown). To center the fastening device inside the blood vessel (as shown in FIG. 20B) relative to an imaginary central axis C thereof, the sheath is removed from the upper portion 206 of the positioning means 201, which is thus expanded to a diameter D_{PM}, as shown in FIG. 20C, thereby pushing to the center of the blood vessel the lower portion 207 of the positioning means still covered by the sheath, thereby preventing the fastening device 203 and particularly its carrier from expanding. The length L_{PM} of the positioning means 201 should be large enough to position the fastening device 203 in the exact place while creating a certain angle β with the blood vessel wall W. In addition, referring to FIG. 20C, the diameter D_{PM} of the positioning means 201 at their upper 206, expanded portion should be larger than the diameter D_{C} of the carrier 205 to ensure the centering of the fastening device 203 inside the blood vessel.

FIGS. 21A and 21B show another example of a positioning means 211 comprising a removable sheath 212 (shown in FIG. 21A in dotted line) for keeping it in its folded state, and a guide wire 214 for first delivering the positioning means and then (after the positioning of the fastening device) removing it from the blood vessel. The positioning means 211 is connected to a fastening device 213 to be centered inside the blood vessel by the guide wire 214. The positioning means 211 is a self-expandable structure i.e. adapted to expand after the removal of the sheath 212. Once the sheath 212 is removed, the expansion of the positioning means 211 causes it to be positioned in the center of the blood vessel (FIG. 21B). As a result, the fastening device 213 is also positioned in the center of the blood vessel. The positioning means has preferably a symmetric structure, adapted not to block the blood flow when in expanded state. It can be made of longitudinal struts, which can be a collection of elastic wires made of materials such as Nitinol, held together at the upper (211a) and the lower (211b) ends of the positioning means 211. Alternatively, the positioning means 211 may be made of longitudinal and transverse braided wires. The positioning means 211 may further comprise a protection cover 216 (FIG. 21B) to avoid harming the blood vessel wall W while expanding and emboli releasing. Other means such as nets, filters or features spread on the external surface of the protection cover 216 may be added to prevent free embloy motion. After the positioning of the fastening device 213 is completed, the sheath 212 is returned and folds back the positioning means 211 and the protection cover 216 to their folded state and they are removed from the blood vessel.

### Bringing the Fastening Device to a State Ready for Penetration of the Blood Vessel

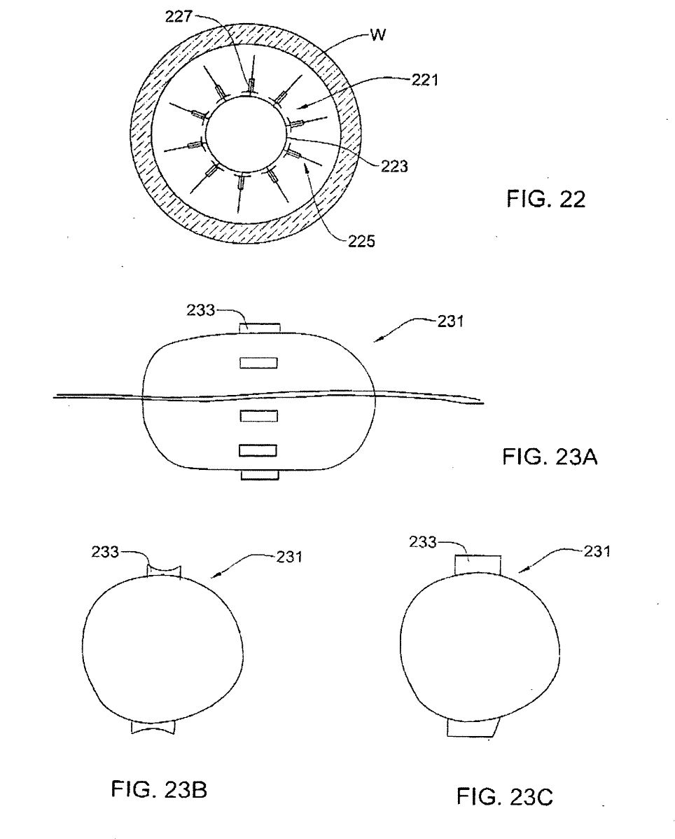

Once the fastening device is positioned inside the blood vessel, the fastening elements are required to be in their penetration state and need to be directed in the penetration direction, i.e. directed radially to the imaginary axis C of the blood vessel. As already explained above, the fastening device is delivered to its desired position inside the blood vessel in the folded state, wherein the fastening elements are not parallel to the penetration direction. Bringing the fastening elements to the penetration direction is performed by removing the external sheath that was covering the fastening device, directly or indirectly, during its insertion into the blood vessel, as explained above. FIG. 22 shows a fastening device 221 comprising a stent graft carrier 223, fastening elements 225 in their penetration state positioned in the center of a blood vessel, whose wall W it suppose to penetrate, and oriented in the penetration direction. The fastening elements are shown with holders 227, which may be any of the holders described in accordance with the present invention, described above, e.g. such as the holder shown in FIG. 4A.

### Penetration Process

During the penetration process the carrier is expanded (as showed e.g. in FIG. 2) by means previously explained, depending on the kind of the carrier, and the fastening elements connected thereto penetrate the wall of the blood vessel. When the carrier is self-expanded, the penetration proceeds by itself. However, in case when the carrier is expanded in its plastic region, such as the carriers 171 of FIGS. 17A to 17E, its expansion may be performed by a balloon. FIGS. 23A to 23C show one example of a balloon suitable to inflate said carriers. FIG. 23A shows a balloon 231 with voluminous forms 233 thereon, which are adapted to be in an deflated (FIG. 23B) or inflated (FIG. 23C) state. The forms 233 are adapted to push the carrying portions 175 of the carrier 171 showed in FIGS. 17A to 17E thereby bending their form from a concave to a convex one. The forms 233 can be inflated by either the same inflating system of the balloon 231 or by a separate system. The described above balloons may be symmetric with respect to some or all of their axes.

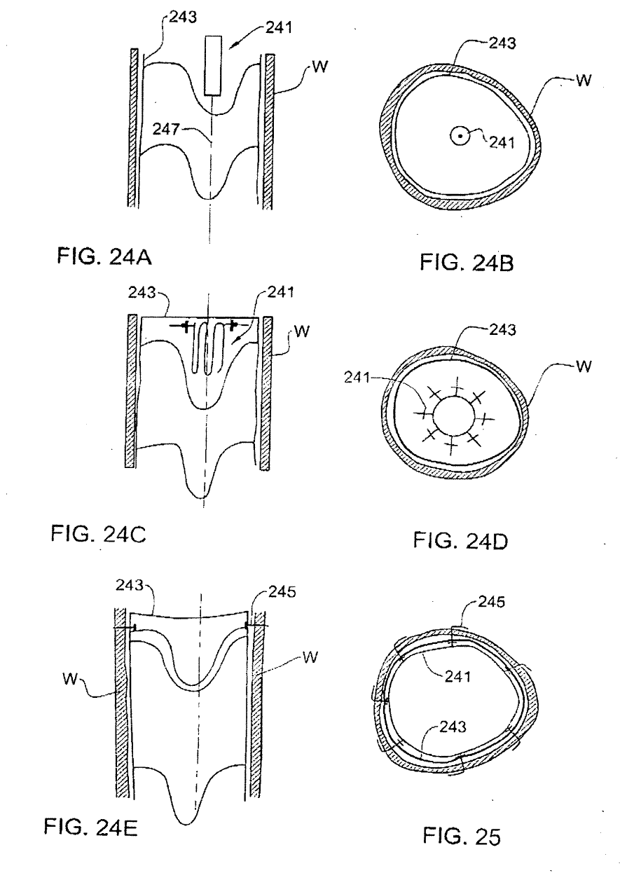

Before explaining the final stage of the present process, it should be reminded again that the fastening device is not necessary an integral part of the stent graft to be fastened. When the carrier is not a part of the stent graft, the fastening device comprising both the carrier and the fastening elements, is inserted into the blood vessel after the stent graft has already been appropriately positioned therein. FIGS. 24A to 24E show the above described stages 1-4 of the present process according to the latter case. FIG. 24A shows a fastening device 241 in its folded state delivered on a guide wire 247 into the blood vessel in which a stent graft 243 has already been positioned. FIG. 24B shows the fastening device 241 delivered to and positioned in its appropriate location in the center of the blood vessel. Next, the sheath (not shown) is removed and the fastening device 241 is ready for penetration (FIGS. 24C and 24D). During the penetration process, as shown in FIG. 24E, the carrier 243 is expanded and the fastening elements 245 penetrate first the stent graft and then blood vessel wall W.

During the penetration process some locking means may be used in order to lock fastening elements 245 to the stent graft.

### Attachment of the Stent Graft to the Blood Vessel

As explained in some of the examples described above, where fastening elements include restraining means for holding their restrainable portions restrained, the attachment of the stent graft takes place once the restraining means, are released, allowing the securing portions of the fastening elements to take their attachment orientations, such as shown in FIGS. 4B, 4C, 5B, 5C, 6B and 25. The restraining means are released either due to the force applied thereon by the blood vessel wall that is penetrated by the fastening elements, such as for example in FIG. 25, or as explained with reference to FIGS. 17A to 19C, due to the change in the form of the carrier or by the rotating means, in which case additional means, such as balloon, may be needed, as previously discussed.

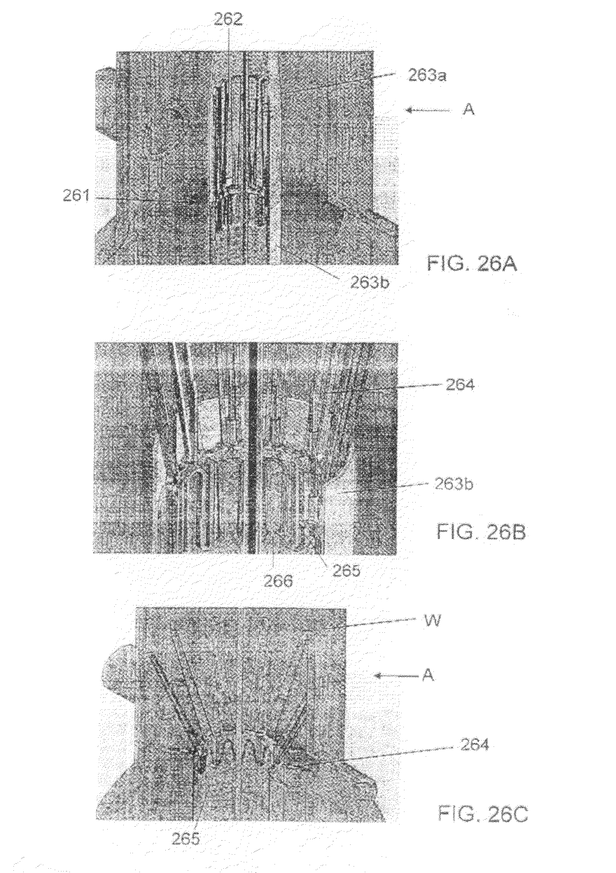

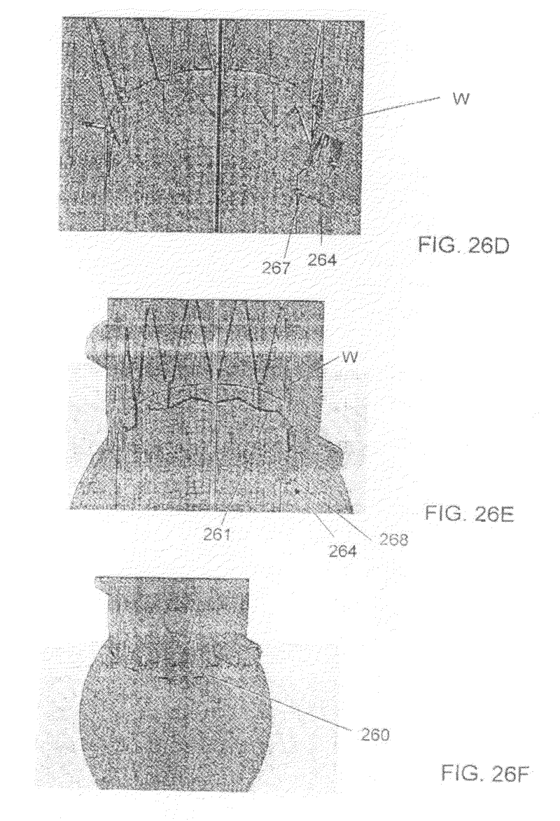

FIGS. 26A to 26F summarize the process according to the present invention, described above. A fastening device 261 is inserted to the aorta A in its folded state, together with positioning means 262 mounted thereon (FIG. 26A). The fastening device 261 and the positioning means 262 are held in their folded state with upper (263a) and lower (263b) sheaths respectively. The upper sheath 263a is removed to allow the positioning means 262 to position the fastening device 261 in the desired place inside the aorta A. As shown in FIG. 26B, the fastening elements 264 and the stent carrier 265, attached to a graft 266, are still kept in the folded state by the lower sheath 263b. Next, the lower sheath 263b is partly removed to allow the fastening elements 264 in their penetration orientation to be in the penetration direction, and then completely removed, as shown in FIG. 26C. The fastening elements 264 are already in the penetration direction, and the carrier 265, as being self-expanded, begins to expand toward the wall W of the aorta. After reaching the wall of the aorta W, the fastening elements 264 begin to penetrate therethrough (FIG. 26D). Due to the force applied by the aorta wall W on the holders 267 that restrain the fastening elements 264, the holders release the restrainable portions of the fastening elements 264 and the attachment is achieved, as the securing portions 268 of the fastening elements 264 tale their attachment orientation (FIG. 26E). A balloon (not shown) may be inflated inside the fastening device 261 to ensure that every fastening element 264 is properly fixed and that it has shifted to its attachment state. The final results are shown in FIG. 26F.

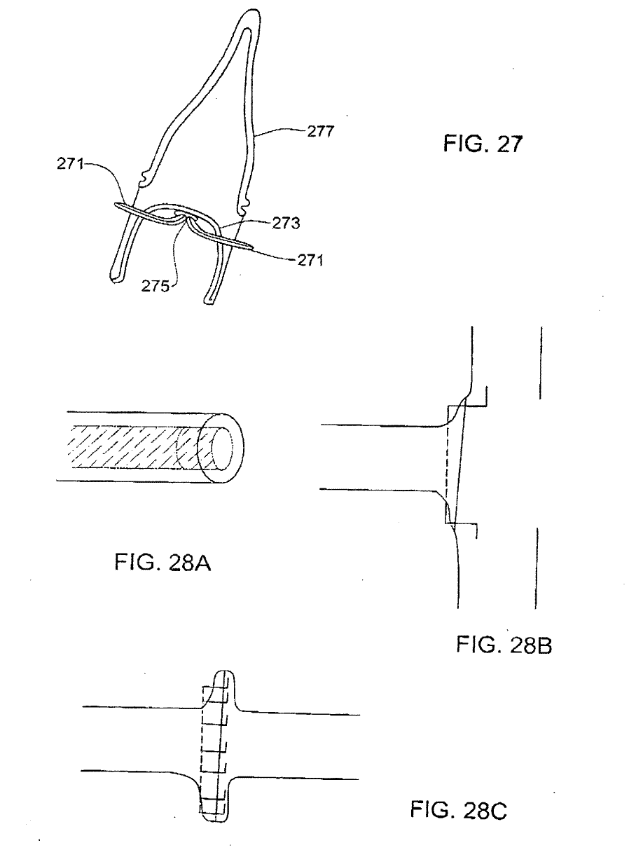

As seen in FIG. 26F, due to the fact that the fastening device described above has a plurality of fastening elements, during the attachment of the stent graft to the aorta a circular connection line 260 is formed by the securing portions in their securing orientation, the connection line constituting a kind of a seam and the securing portions constitute a kind of stitches thereof. In general, depending on the length of the securing portions, the direction in which they extend in their securing orientation (which may be different from that shown in FIG. 26F, as illustrated for example in FIGS. 11 and 17E), and the mutual disposition between the fastening elements, as for example shown in FIG. 10B, in the fastening device, the connection line may have a desired configuration and, for example, may be more or less continuous. Spacing between adjacent stitches may not be uniform and they may be arranged in couples, e.g. such as shown in FIG. 27, where fastening elements 271 and holder 275 are used of the kind described above with reference to FIGS. 4C and 7A.

FIG. 27 also shows one example of how a fastening device may be formed integrally with a stent, whose upper portion 277 forms a positioning means (similarly to that shown in FIG. 20A) and lower portion 273 constitutes a carrier of the fastening device.

In the process described above example was used of a side to side attachment (FIG. 28A) of a stent graft to a blood vessel. However, the process and devices of the present invention may be applicable to other cases, including those where tubular objects are attached to each other in dispositions shown in FIG. 28B (end to side attachment) and FIG. 28C (end to end attachment). Those skilled in the art to which this invention pertains will readily appreciate that numerous changes, variations, and modifications can be made without departing from the scope of the invention, mutatis mutandis.

1-66. (canceled)

67. A fastening device to be associated with a first object, for the attachment of said object to a second object, the device comprising at least one fastening element having a base portion and at least one securing portion, said fastening element being originally produced with its securing portion having a first orientation relative to the base portion and with a possibility to bring the securing portion into a second orientation relative to the base portion, to allow said at least one securing portion to penetrate said second object being exposed thereto while staying in said second orientation, and being adapted to change said second orientation into said first orientation to provide said attachment.

68. A fastening device according to claim 67, wherein said at least one fastening element comprises a restrainable portion between the base and the securing portions thereof, said fastening element being originally produced with its securing portion having said first orientation relative to the base portion and with the possibility to bring the securing portion into said second orientation relative to the base portion by restraining said restrainable portion so as to enable the securing portion to return from its second orientation to its first orientation when the restraint is released from the restrainable portion; the fastening element in said device having a penetration state in which said restrainable portion is restrained to keep said at least one securing portion in said second orientation constituting its a penetration orientation, to allow said at least one securing portion to penetrate said second object while staying in said penetration orientation, and being adapted to change said penetration state to an attachment state in which said restraint is released to let said at least one securing portion return to its said first orientation, constituting an attachment orientation thereof at which the first object is attached to the second object.

69. A fastening device according to claim 68, being a medical fastening device adapted for use with a stent.

70. A fastening device according to claim 69, wherein said stent comprises a stent graft and said device further comprises a carrier with at least one carrying portion on which said at least one fastening element is mounted, the stent graft having at least one connection area, said carrier being insertable into the interior of the stent to allow said fastening element to penetrate said stent graft at, and to project from, said connection area.

71. A fastening device according to claim 69, constituting an integral part of said stent, the stent constituting a carrier having at least one carrying portion on which said at least one fastening element is mounted.

72. A fastening device according to claim 67, further comprising a carrier with at least one carrying portion to which more than one said fastening element is mounted.

73. A fastening device according to claim 70, wherein the device comprises a plurality of fastening elements and a plurality of restraining elements each for keeping the restraining portion of at least one fastening element in the restrained state, each of said restraining elements being adapted for applying thereto of an actuating force for releasing the restrainable portions and thereby bringing the securing portions of the fastening elements into their attachment orientation.

74. A fastening device according to claim 73, wherein said carrier is provided with an arrangement adapted to apply to each said restraining element said actuating force upon the carrier having expanded to a predetermined extent defined by the length of said at least one securing portion.

75. A fastening device according to claim 73, wherein at least one of said restraining elements is movable relative to said fastening element between a restraining position in which the restraining element applies, directly or indirectly, a restraining force to the restrainable portion to keep said at least one securing portion in said penetration orientation, and a releasing position to remove said restraining force from said restrainable portion, thereby bringing the at least one securing portion to said attachment orientation.

76. A fastening element according to claim 73, wherein said restraining element has a flange disposed at its proximal end and laterally projecting from the restraining element, and is adapted to move upon the application to its flange of a force directed away from said securing portion, said force constituting said actuating force.

77. A fastening element according to claim 76, wherein said flange is disposed at the restraining element's proximal end and laterally projects from the restraining element.

78. A fastening device according to claim 68, wherein said fastening element is a single body elastically deformable at least at said restrainable portion.

79. A fastening device to be associated with a first object, for the attachment of said object to a second object, the device comprising at least one fastening element having a base portion, at least one securing portion and a restrainable portion between the base portion and the at least one securing portion, the fastening element having a penetration state in which said restrainable portion is restrained to keep said at least one securing portion in a penetration orientation relative to the base portion such as to allow said at least one securing portion when in said penetration orientation to penetrate said second object while staying in said penetration orientation, and being adapted to change said penetration state to an attachment state in which said restrainable portion is released to bring said at least one securing portion into an attachment orientation different from the penetration orientation, wherein the attachment state of the fastening element is at least similar to an initial state of the fastening element, which it had before the restrainable portion was restrained, and wherein the device further comprises at least one restraining element mounted on at least said restrainable portion for keeping the restraining portion in the restrained state, the restraining element being adapted for applying thereto of an actuating force for releasing the restrainable portion and thereby bringing the securing portion of the fastening elements into their attachment orientation.

80. A fastening device according to claim 79, wherein the restraining element is movable relative to said fastening element between a restraining position in which the restraining element applies, directly or indirectly, a restraining force to the restrainable portion to keep said at least one securing portion in said penetration orientation, and a releasing position to remove said restraining force from said restrainable portion, thereby bringing the at least one securing portion to said attachment orientation.

81. A fastening device for the attachment of a first object to a second object, the device comprising a carrier having at least one carrying portion and at least one fastening element mounted to said carrying portion so as to be stationary relative thereto, the carrier constituting, or being connectable to, said first object, said at least one carrying portion of the carrier being plastically deformable to change its initial state in which the fastening element is directed in a penetration direction to penetrate said second object, to its final state in which the fastening element is directed transversely to said penetration direction to at least partially press said second object towards said carrier.

82. A stent adapted for being deployed in a blood vessel and having at least one connection area, the stent being adapted, at least after having been deployed in the blood vessel, to function as a fastening device and to comprise at least one fastening element projecting from said connection area in a penetration direction and having a securing portion capable of changing its orientation relative to said penetration direction between a penetration orientation allowing at least said securing portion to penetrate the blood vessel's wall, and an attachment orientation in which the securing portion is oriented transversely to the penetration direction and is adapted to at least partially press said vessel's wall towards said connection area of the stent.

83. A stent according to claim 82, further comprising a graft, constituting together a stent graft, said stent-graft being adapted to receive therein a fastening device comprising a carrier and a plurality of the fastening elements adapted to penetrate said stent graft at a connection area thereof before the penetration of said blood vessel's wall.

84. A stent according to claim 70, wherein said carrier has carrying portions to which said fastening elements are mounted, said carrying portions being adapted to change their shape and/or orientation relative to adjacent portions of the carrier, thereby changing special disposition of the fastening elements between their penetration disposition at which the securing portions thereof have said penetration orientation and said attachment orientation in which the securing portion is oriented transversely to the penetration direction.

85. A method for producing a fastening device to be associated with a first object, for the attachment of said object to a second object, the device comprising at least one fastening element having a base portion and a securing portion capable of changing its orientation relative to the base portion between a penetration orientation in which the securing portion is adapted to penetrate said second object, and an attachment orientation transverse to the penetration orientation, said method comprising:
producing said fastening element with said securing portion in an initial orientation at least similar to the attachment orientation with a possibility of changing said initial orientation to a penetration orientation when a restraining force is applied thereto; and
placing a restraining element on the fastening element so as to apply thereto said restraining force to bring the securing portion in said penetration orientation, said restraining element being movable in a direction along the fastening element, to remove said restraining force from the securing portion, thereby bringing the securing portion in said attachment orientation, the restraining element having an dimension in said direction shorter than the fastening element.

86. A method for the attachment of a first object to a second object, comprising:
providing at least one fastening element extending in a penetration direction, projecting from or connected to said first object, and having a securing portion, the fastening element being adapted to change its state between a penetration state in which the securing portion has a penetration orientation suitable to penetrate said second object, and an attachment state in which said securing portion has an attachment orientation transverse to the penetration orientation, wherein said attachment orientation of said securing portion is a predetermined state with which said fastening element was originally produced with a possibility to bring the securing portion into the penetration orientation by applying to the fastening element a restraining force and to enable the securing portion to return from its second orientation to said predetermined state when the restraining force is withdrawn;
causing at least the securing portion while in the penetration orientation, to penetrate said second object; and
withdrawing said restraining force to have the fastening element change its state to said attachment state.

87. A method according to claim 86, comprising providing a plurality of the fastening elements, whose securing portions, when in said attachment orientation, are aligned to form a seam on or in said second object.

88. A method according to claim 87, wherein said fastening element is bendable, the method further comprising changing the state of said fastening element from a non-bent state in which said securing portion is in said penetration orientation, into a bent state in which said element is bent at said bending area and said securing portion is in said attachment orientation.

89. A method according to claim 88, further comprising setting a predetermined bent state to said fastening element by a bending force, before bringing it to said non-bent state.

90. A method according to claim 88, further comprising keeping said at least on fastening element in said non-bent state by providing a compensating force against said bending force by at least one holder, and allowing the bending force to bend the fastening element at said bending area by moving of said at least one holder relative to the at least one fastening element.

91. A method according to claim 86, further comprising keeping said at least one fastening element in said attachment orientation by a locking means.

92. A method according to claim 90, further comprising providing a plurality of the fastening elements mounted on a common carrier.

93. A method according to claim 92, wherein said first and second objects are tubular objects of which the first object is deployed with the second object.

94. A method according to claim 93, wherein said carrier is a part of the first object.

95. A method according to claim 93, wherein said fastening device is deployed within the first object, and said projecting of the fastening elements is achieved by the fastening elements penetrating said first object prior to their penetration of the second object.

96. A method according to claim 94, wherein the carrier is originally in a collapsed state with said fastening elements in their penetration state being directed in a direction different from the penetration direction, the method further comprising essentially simultaneously aligning the fastening elements in the penetration direction.

97. A method according to claim 96, wherein the carrier is capable of collapsing and expanding, and the aligning of the fastening elements in the penetration direction is performed before the carrier is in its expanded state, the expansion causing the fastening elements to penetrate the second object.

98. A method according to claim 97, wherein the collapsed state of the carrier is obtained by exerting a compression force on said carrier by a sheath.

99. A method according to claim 98, wherein the expansion and the collapsing of the carrier is performed by its respective inflation and deflation by a balloon.

100. A method according to claim 99, wherein upon the penetration of the second object to a predetermined extent, further expansion of the carrier causes release of said restraining force, changing thereby the orientation of the securing portions of the fastening elements from the penetration orientation to the securing orientation.

101. A method according to claim 94, wherein upon the penetration of the second object until its contact with said holder, further expansion of the carrier causes the holder to move relative to fastening element, thereby releasing said restraining force.

102. A method according to claim 93, further comprising positioning means for the deployment of the fastening device into a desired position within the second object.

103. A method according to claim 93, further comprising centering said fastening device relative to the axis of said second object.
